(19) United States
(12) Patent Application Publication
(10) Pub. No.: US 2020/0400694 A1
(43) Pub. Date: Dec. 24, 2020

(54) METHOD FOR ASSISTING DETERMINATION OF RISK OF CARDIOVASCULAR DISEASE OR THE LIKE

(71) Applicant: Denka Company Limited, Tokyo (JP)

(72) Inventor: Yasuki ITOH, Gosen-shi (JP)

(73) Assignee: Denka Company Limited, Tokyo (JP)

(21) Appl. No.: 16/979,118

(22) PCT Filed: Mar. 13, 2018
(86) PCT No.: PCT/JP2018/009719

§ 371 Date: Sep. 8, 2020

(51) Int. Cl. G01N 33/92

(52) U.S. Cl. CPC G01N 33/92; G01N 2800/32

(57) ABSTRACT

Disclosed are a method of assisting in determination of risk of cardiovascular disease, coronary heart disease, or cerebral stroke, and a method of assisting in diagnosis of cerebral stroke. The method of assisting in determination of risk of cardiovascular disease, coronary heart disease, or cerebral stroke includes measuring LDL-TG value in blood isolated from a biological body, and a high measured LDL-TG value indicates a high risk of cardiovascular disease, coronary heart disease, or cerebral stroke. The method of assisting in diagnosis of cerebral stroke includes measuring LDL-TG value in blood isolated from a biological body, and a high measured LDL-TG value indicates a high possibility of onset of cerebral stroke.

### TECHNICAL FIELD

The present invention relates to a method of assisting in determination of risk of cardiovascular disease, coronary heart disease, or cerebral stroke, and to a method of assisting in diagnosis of cerebral stroke.

### BACKGROUND ART

Cholesterol, triglycerides, phospholipids, and the like are major components of serum/plasma lipids, and the blood lipids are associated with apoproteins to form lipoproteins, which circulate in blood. These lipoproteins are classified into the following categories, on the basis of their density, including, for example, chylomicrons (CM), very low density lipoproteins (VLDL), intermediate density lipoproteins (IDL), low density lipoproteins (hereinafter also referred to as LDL), and high density lipoproteins (hereinafter also referred to as HDL). Among those lipoproteins, LDL is a major transporter that transports cholesterol from the liver to tissues, and increased levels of LDL cholesterol (hereinafter also referred to as LDL-C) have been described to have close relation with development of arteriosclerosis. For this reason, LDL-C is believed to be a risk factor for arteriosclerosis, ischemic heart disease (coronary artery disease), and the like, and measurement of the content of LDL-C is considered as an important indication for diagnosis, treatment, and prevention of these diseases. Meanwhile, ischemic heart disease and the like have been found to occur in many cases even though the LDL-C level is within the normal level in the blood. Thus, much attention has recently been focused on changes in the quality of LDL particles.

LDLs containing high amounts of triglycerides (hereinafter also referred to as TG-rich LDL) are lipoproteins with properties different from those of normal LDLs with high cholesterol content, and are often observed in blood from patients with liver disease, and are reported to be increased in concentration in the blood as the liver disease progresses and to account for the majority of lipoproteins present in the blood in the terminal stage of liver disease. Moreover, there are reports: the rate of transformation of macrophages into foamy macrophages, which is induced by TG-rich LDL, is directly proportional to the concentration of malondialdehyde-modified LDL, an oxidized LDL, in serum; a significant increase of triglyceride hydroperoxides is observed in blood from patients with liver disease though almost no triglyceride hydroperoxides are detected in blood from healthy individuals; and the like. From these reports, triglycerides in LDL are also considered to have close relation with oxidized LDLs. The blood LDL-TG level is reported to be higher in patients with coronary heart disease, when triglycerides in LDL (hereinafter also referred to as LDL-TG) are compared between patients with coronary heart disease (CI ID) and healthy individuals (Non-Patent Document 1). However, no evidence is currently available to indicate that the risk to develop coronary heart disease in the future is increased in healthy individuals who have not developed coronary artery disease but show a high LDL-TG level.

On the other hand, no evidence is available to indicate the relationship between cerebral stroke and LDL-TG, and it has not been clear whether or not the LDL-TG value can be used to diagnose patients with cerebral stroke, or whether or not the risk to develop cerebral stroke in the future is increased in test subjects with a high LDL-TG value.

In a method of measuring the amount of LDL-TG, two operational steps of fractionation and triglyceride quantification are combined for the measurement. The fractionation operation includes methods in which ultracentrifugation, electrophoresis, and/or high-performance liquid chromatography are utilized, while the quantification method includes a method in which the quantification is performed using, for example, an automatic analyzer together with a reagent for triglyceride measurement, which are used in clinical settings. Although the combination of both the operations enables measurement of LDL-TG, the method is complicated and time-consuming because the method is carried out by two steps, that is, a pretreatment step to separate LDL completely from lipoproteins other than LDL and a step to perform measurement. Moreover, depending on the fractionation method, simple or quantitative recovery of isolated samples themselves is difficult. Furthermore, even a method that allows quantitative recovery may require skills or special instruments for the operation. These methods cost high, and are unpopular due to the inconvenience and high cost.

As methods to solve the above problems and to allow the above measurement on, for example, an automatic analyzer without performing the fractionation operation, a method in which triglycerides in all the lipoproteins other than LDL are removed in the first step and the amount of triglycerides in the remaining LDL is then measured in the second step (Patent Documents 1 and 2), and a method in which (free glycerol and) triglycerides in HDL are removed in the first step and only triglycerides in LDL are then measured in the second step (Patent Document 3) are known.

### PRIOR ART DOCUMENTS

### Patent Documents

Patent Document 1: JP 2006-180707 A
Patent Document 2: US2005/042703 A1
Patent Document 3: US2015/132834 A1

### Non-Patent Document

Non-Patent Document 1: Circulation 2004; 109, 2844-2849

### SUMMARY OF THE INVENTION

### Problems to be Solved by the Invention

An object of the present invention is to provide a method of assisting in determination of risk of cardiovascular disease, coronary heart disease, or cerebral stroke. Another object of the present invention is to provide a method of assisting in diagnosis of cerebral stroke.

The present inventors intensively studied to find that the risk to develop cardiovascular disease, coronary heart disease, or cerebral stroke in the future is increased in healthy individuals with a high blood LDL-TG value but without cardiovascular disease, coronary heart disease, or cerebral stroke, and that the blood LDL-TG value is high in patients with cerebral stroke, to complete the present invention.

### Means for Solving the Problem

That is, the present invention provides the following:

(1) A method of assisting in determination of risk of cardiovascular disease, coronary heart disease, or cerebral stroke, comprising measuring LDL-TG value in blood isolated from a biological body, wherein a high measured LDL-TG value indicates a high risk of cardiovascular disease, coronary heart disease, or cerebral stroke.
(2) A method of assisting in diagnosis of cerebral stroke, comprising measuring LDL-TG value in blood isolated from a biological body, wherein a high measured LDL-TG value indicates a high possibility of onset of cerebral stroke.

### Effects of the Invention

The present invention provides, for the first time, a method to correctly determine the degree of risk for the future development of cardiovascular disease, coronary heart disease, or cerebral stroke before onset of any of these diseases. Those who are determined to have a high-risk disease could make efforts, such as changing lifestyle or taking medication, to reduce the risk of the disease, and thereby prevent the onset of the disease.

Additionally, the present invention can assist in quick and exact diagnosis of cerebral stroke, which thus enables prompt treatment of cerebral stroke in the early stage and prevention of cerebral stroke-related death.

### MODE FOR CARRYING OUT THE INVENTION

In the methods of the present invention, the blood LDL-TG value is measured. Serum or plasma is preferably used as a blood sample for convenience in measurement.

A method of measuring the blood LDL-TG value per se is well known (for example, Patent Documents 1 to 3), and reagents for the measurement are commercially available. Thus, the amount of LDL-TG in the blood can easily be measured using the commercially available reagents. Briefly, the LDL-TG value can be measured by a method, such as a method (1) in which a sample is treated with a surfactant that acts on lipoproteins other than LDL (such as, for example, a polyalkylene oxide derivative with an HLB value of not less than 13 and not more than 14) and then treated with a lipoprotein lipase, a glycerol kinase, and a glycerol-3-phosphate oxidase in the presence of a surfactant that acts on LDL (such as a polyalkylene oxide derivative with an HLB value of not less than 11 and less than 13) to measure the amount of the produced hydrogen peroxide, or a method (2) in which a sample is treated with a lipoprotein lipase, a cholesterol esterase, a glycerol kinase, and a glycerol-3-phosphate oxidase in the presence of a surfactant that acts on lipoproteins other than LDL (such as, for example, a polyalkylene oxide derivative with an HLB value of not less than 13 and not more than 15), followed by removal of the produced hydrogen peroxide, and then treated with a lipoprotein lipase, a glycerol kinase, and a glycerol-3-phosphate oxidase in the presence of a surfactant that acts on LDL (such as a polyalkylene oxide derivative with an HLB value of not less than 11 and less than 13) to measure the amount of the produced hydrogen peroxide. In the Example below, the quantification is performed using a commercially available LDL-TG quantification kit (LDL-TG “Seiken”; manufactured by Denka Seiken Co., Ltd.).

Among healthy individuals without cardiovascular disease, coronary heart disease, or cerebral stroke, those who show a higher blood LDL-TG value are at a higher risk to develop these diseases in the future. Thus, the measurement of blood LDL-TG value can assist in determination of the risk to develop those diseases in the future. Because a higher blood LDL-TG value is associated with a higher risk to develop these diseases in the future, a specific evaluation criterion can be appropriately set. In the Example below, for example, test subjects were divided into approximately equally sized quartile groups, on the basis of blood LDL-TG values, to investigate the incidence of each disease within 15.6 years in each group, and the results of the investigation indicated that the incidence of each disease was statistically significantly increased and a statistically significant difference in incidence was found in each of the second, third, and fourth quartile groups, as compared to the baseline incidence in the first quartile group, which showed the lowest LDL-TG value. Because the second quartile has a lower limit value of 17.1 mg/dL in this respect, the value of 17 mg/dL may be set as a threshold, for example, to determine a value above 17 mg/dL as an indicator of high-risk disease. Alternatively, in cases where a higher level of risk is to be identified, for example, a value not less than the median value of 22.7 mg/dL can also be used as an indicator of high-risk disease. As seen above, the threshold value can be arbitrarily set in consideration of the degree of detected risk. For example, an arbitrary value within the range from 17 mg/dL to 40 mg/dL, particularly within the range from 25 mg/dL to 35 mg/dL, may be set as a threshold. Since the LDL-TG value may vary according to characteristics of a population, such as race, the threshold value may be, for example, the median, the mean, or the boundary value between the first and second quartile in the population, or the threshold value may be a value close to any of these values (such as, for example, an arbitrary value within any of these values±20%), with which the evaluation can be made. Furthermore, because the risk of each disease is increased in response to elevation of LDL-TG value above an appropriately set threshold value, the risk of each disease can be estimated based on specific measured values, and the probability of each risk can be expressed numerically by relating each measured value to the occurrence frequency of each event.

Similarly, blood LDL-TG value can be involved in assistance in diagnosis of cerebral stroke in onset, which can further evaluate or determine the severity of the cerebral stroke. For example, a measured LDL-TG value of 20 to 40 mg/dL, preferably more than 30 mg/dL, is determined to indicate a higher risk to develop CVD, CHD, or cerebral stroke in the future. However, the reference value for FCHL is not restricted to these values because the reference value is thought to vary depending on, for example, race. Moreover, even among individuals diagnosed to have a high risk to develop CHD or cerebral stroke, it can be judged that the risk to develop each disease is increased in those who have an increasing LDL-TG value, such as 40 mg/dL, 50 mg/dL, and so on.

The present invention will be specifically described below by way of an example. However, the present invention is not limited to the example below.

### Example 1

The concentration of LDL-TG was measured using stored samples (serums) from a cohort study conducted in the United States. Specifically, the concentration of LDL-TG was measured in 9,334 subjects without CHD or cerebral stroke at the time of baseline blood collection. Then, the test subjects in the cohort study were followed for 15.6 years to investigate the incidence of CHD or cerebral stroke (and CVD as a combination of these diseases), and the correlation between the measured item and the incidence of each disease was examined.

LDL-TG was measured by using LDL-TG “Seiken” (manufactured by Denka Seiken Co., Ltd.).

The LDL-TG values were divided into quartile groups, and the COX proportional regression model was used to obtain hazard ratios with respect to the first quartile group.

The results are presented in Table 1.

TABLE 1

| | | LDL-TG (mg/dL) | | | | |
|---|---|---|---|---|---|---|
| | | Q1 | Q2 | Q3 | Q4 | P trend of |
| | | (0.7-17) | (17.1-22.6) | (22.7-29.6) | (29.7-104) | linearity |
| CHD | n/N | 257/2360 | 326/2342 | 403/2301 | 448/2331 | <0.001 |
| | (%) | (10.89) | (13.92) | (17.51) | (19.22) | |
| | HR | 1.0 | 1.30 | 1.69 | 2.02 | <0.0001 |
| | 95% CI | | (1.10-1.53) | (1.45-1.98) | (1.73-2.36) | |
| cerebral | n/N | 98/2360 | 123/2342 | 101/2301 | 161/2331 | <0.001 |
| stroke | (%) | (4.15) | (5.25) | (4.39) | (6.91) | |
| | HR | 1.0 | 1.34 | 1.13 | 1.85 | <0.0001 |
| | 95% CI | | (1.03-1.75) | (0.85-1.49) | (1.43-2.38) | |
| CVD | n/N | 327/2360 | 420/2342 | 472/2301 | 566/2331 | <0.001 |
| | (%) | (13.86) | (17.93) | (20.51) | (24.28) | |
| | HR | 1.0 | 1.35 | 1.59 | 2.04 | <0.0001 |
| | 95% CI | | (1.17-1.56) | (1.38-1.83) | (1.78-2.34) | |

As seen in Table 1, the hazard ratios of CHD, cerebral stroke, and CVD with respect to the first quartile group were increased in each of the second, third, and fourth quartile groups, which are based on the LDL-TG values, indicating that the risk of each disease was increased in each of the former quartile groups. The above results indicate that measurement of LDL-TG value enables determination of the degree of risk for the future development of CHD or cerebral stroke (and CVD as a combination of these diseases).

1. A method of assisting in determination of risk of cardiovascular disease, coronary heart disease, or cerebral stroke, comprising measuring LDL-TG value in blood isolated from a biological body, wherein a high measured LDL-TG value indicates a high risk of cardiovascular disease, coronary heart disease, or cerebral stroke.

2. A method of assisting in diagnosis of cerebral stroke, comprising measuring LDL-TG value in blood isolated from a biological body, wherein a high measured LDL-TG value indicates a high possibility of onset of cerebral stroke.
